(19) United States
(12) Patent Application Publication
(10) Pub. No.: US 2023/0000110 A1
(43) Pub. Date: Jan. 5, 2023

(54) Edible Thermal Pressure Molded Composites with Defined Material Characteristics

(71) Applicant: Charles Marsland, Windermere, FL (US)

(72) Inventor: Charles Marsland, Windermere, FL (US)

(21) Appl. No.: 17/943,304

(22) Filed: Sep. 13, 2022

Related U.S. Application Data
(63) Continuation of application No. PCT/US2021/022350, filed on Mar. 15, 2021.
(60) Provisional application No. 62/989,281, filed on Mar. 13, 2020.

(51) Int. Cl. A23J 3/22; A23J 3/10; A23C 9/13; A23C 9/12; A23C 9/18

(52) U.S. Cl. CPC A23J 3/227; A23J 3/10; A23J 3/222; A23C 9/1307; A23C 9/1216; A23C 9/18

(57) ABSTRACT

Edible thermal pressure molded and shaped composites made from the milk of ruminant animals. The composites have combinations of desirable material characteristics. Each composite can be characterized utilizing physical peak compression strength at associated temperature, with specific total moisture.

### CROSS-REFERENCE TO RELATED APPLICATIONS

This application claims priority of International Patent Application PCT/US2021/022350 filed on Mar. 15, 2021, which itself claimed priority of Provisional Patent Application 62/989,281 filed on Mar. 13, 2020 (now expired).

### BACKGROUND

This disclosure relates to meat and seafood substitutes.

The world's population is dramatically increasing, creating a greater need for sustainable protein. In order to accommodate this essential demand for protein, we need to dynamically shift away from high animal meat consumption models to alternative forms. However, many, including plant sources, possess inadequate nutritional profiles of amino acids, need complex ingredient structures to represent a final product and/or are ultra-processed expensive representations of meat including cell culture derived formats. While change occurs, to feed the world, simplicity and safety must become more important as human health and medical research links the food industry's use of unnecessary additives and ingredients to detrimental long term health issues. Increased amyloid plaque in brain tissue, microbiota imbalances, impaired glucose processing, slowed metabolism, leaky gut, immune system chaos, and perpetual inflammation are just some of the resulting effects. To add to that, increased greenhouse gases are now highlighted in climate research since information pertaining to the industrialized world's pollutants becomes everyday news. That compounded with data showing Earth's oceans are contaminated with micro plastics and chemical mixtures too complex to understand, all jeopardize the human protein supply chain. Bottomline, the demands of the human diet aren't helping the planet environmentally. But there are ways we can. For example, using advanced technology and the existing supply chain of protein can in many ways change the carbon footprint. New types of foods that don't rely on industrial food chemicals, fake colors, extreme chemistry, or methodologies that yield unknown long term health results can replace the outdated.

The technology presented herein focuses on the use of ruminant milk to subsidize the increasing need for meat protein structures. However, liquid and powdered ruminant milk and milk proteins are not easy to work with never mind attempt to convert into a meat/seafood substitute which mimics physical and handling characteristics of meat. Liquid and powdered ruminant milk and milk proteins are well known and characterized, but milk proteins gel, which is not a desired feature when making fibrous flesh-like textured edible materials. Casein is the milk protein component involved in these gels. The most prominent example of a casein gel is cheese. Other gels have also been obtained from caseinates, a derivative of casein. However, none of these gels can be utilized in the production of meat and seafood substitutes because they have the disadvantage of being reversible and liquefy on cooling or they melt and liquefy with heat as cheese does. These reversible properties add extreme complexity when developing a new technology that can be used as a meat and seafood substitute.

The main use of milk proteins, including caseins and caseinate, is for their gelation properties and even those are limited. They include binding, thickening and/or emulsifying additives into other food components. The primary use for casein, as a solid and semi-solid gel, is limited to cheese and yogurt. However, there are other products that have been obtained from casein with the addition of gums, cross linking agents or gelling chemicals. This type of pathway leads to structures with a homogenous texture not resembling meat making them commercially undesirable and organoleptically unacceptable to consumer taste and texture requirements.

The food industry is constantly working at altering structure and texture with ingredients. But it does not innovate well. Simple technologies advance such as the use of plant protein analogues with polysaccharide additives. Inclusion of these functional starches such as potato or modified wheat and corn, are being used to increase rigidity and water absorption in fake meat items including surimi gels, plant burgers, fake pork and fake chicken. Additionally gums including carrageenans, alginates, glucomannan, modified cellulose gums (e.g. methyl and carboxymethyl cellulose) are now being used extensively in a wide range of food systems as thickening, suspending, binding, forming and gelling agents. They are frequently added to plant based meats, fake seafoods, dairy products, ice cream, nut milks, syrups, beverages and more to modify viscosity or bind un-bindable components. They are also critical to the developing cell culture meat industry. High percentage concentrations of these gums, particularly alginates, are used to create seafood analogues like fake fish and shrimp.

U.S. Pat. No. 4,880,654 discloses a method for making fibrous particles resembling torn flakes of meat that are prepared by mixing, kneading and beating an aqueous solution of soluble alginate and protein and/or starch with aqueous polyvalent cation gelling agent solution. The fibrous particles are mixed with meat paste and then shaped and cooked to produce a meat analog product having predetermined shape. This technology relies on a high alginate, starch and water composition along with actual meat/flesh to achieve desirable textures. The use of alginate is beneficial but it is an expensive ocean-sourced ingredient. This technology doesn't solve the world's forthcoming needs because it is more a meat extender technology using expensive complex mixtures of ocean sourced alginate gums and high glycemic starches with seafood or animal protein mixed in.

U.S. Pat. Application Publication No. 20180070609 relates to a process for producing a casein protein product by using a casein concentrate (e.g., powder) starting material; heat-treating the material; cooling the heat-treated material; subjecting the cooled material to a treatment with crosslinking agents; and then processing the material into the casein protein product. This technology needs isolated purified casein because the inclusion of the other milk components whey protein, lactose and milk fat weakens this technology's structure and functionality. That rules out the use of ruminant milk as a feedstock, making it not feasible because this technology chemistry is 100% based on pure casein isolate with a cross linking agent. All other natural components make this technology fail.

U.S. Pat. Application Publication No. 20050112271 discloses a meat alternative, having from about 65% to about 85% by weight of a hydrated plant protein source and from about 15% to about 35% by weight of a meat protein source and is formed by mechanically or chemically extracting collagen into a slurry of the meat and water with the collagen supporting the plant protein in the final product to create a texture and flavor. This technology is an animal meat extender.

U.S. Pat. No. 3,873,736 discloses a semi-moist protein food product of meaty and other materials having an elastic texture resembling that of natural meat. The product is made by adding a wheat gluten to a mix of cooked meats and water-soluble substances and then raising the temperature to coagulate the protein and form a solid product. The use of gluten is primary to the texture and viscoelasticity of the material. It is also heavily based on the inclusion of humectants including glycerol, propylene glycol, sorbitol for added moistness of which none are desirable.

U.S. Pat. No. 5,368,871 discloses a formulation and process for making seafood analogs. It utilizes a very high level of the chemical sodium hexametaphosphate (SHIVIP) as well as kappa carrageenan gum. The product claims thermal stability but that may be from the high level of SHMP which is used as a protein bridging agent. This product would be considered toxic in today's regulated food world.

U.S. Pat. No. 4,209,534 discloses a method for producing a thermally stable textured milk protein product resembling beef. The process involves many complicated and environmentally difficult and unacceptable food processing steps such as hydrochloric acid hydrolysis followed by polyphosphate treatment, which is now linked to deadly amyloid plaque formation in human organs (e.g. brain and liver), followed by lyophilization which is prohibitively expensive batch freeze drying. For meat color it utilizes food coloring agents which are also highly undesirable in todays limited food ingredient world.

U.S. Pat. No. 3,996,390 discloses a system for the preparation of an acidified milk gel. The composition comprises a thickener system of carboxymethyl cellulose and gelatin of which both are chemically and ideologically undesirable. Carboxymethyl cellulose is an environmentally harmful low level thickener/gum and gelatin is an expensive animal waste byproduct protein made from animal and fish bones, cartilage, and skin. This patent uses undesirable ingredients and is considerably outdated.

U.S. Pat. No. 2,813,794 discloses a process for preparing a chewy casein gel for use in protein food products simulating meat however only has one 50 gram example. This single process example represents a small scale lab experiment.

U.S. Pat. No. 4,250,198 discloses a casing stuffed meat snack analog using potassium sorbate and sodium nitrite, synthetic coloring agents such as “sunset yellow”, and undesirable MSG. It is an example of heavy use of outdated and now considered dangerous food ingredients. Because of that this type of food is not considered edible in today's food world.

### SUMMARY

Aspects and examples are directed to stable edible meat/seafood substitute composite materials made from the milk of ruminants. These composite materials can be molded into shapes that are like the shapes of common meats and seafoods, such as a thick, generally round slab that looks like a filet mignon, or a small round piece that looks like a scallop, or a generally oval piece that looks like a chicken breast. as just three of many possible examples. Molding can take place in a silicone mold that is able to stand up to the molding pressures and temperatures, and has the desired shape; the mold shape is imparted to the final composite material. The final compression strength can be designed to mimic that of the meat or seafood that is being substituted. The compression strength of the materials have been found to be controllable by adjusting the relationship of heat, pressure and time parameters. The increased parameters yield different characteristics than the lesser, therefore showing a distinct technology which allows for a unique algorithm with the materials being processed. When desired properties of the final product are low compression, low pressure and low temperature are utilized for setting the material characteristic's desired parameters. For higher compression strength, the temperature and pressure parameters are adjusted upward to achieve the desired material characteristics. Both extremes in conjunction with controlled final water content are relevant to the final product quality, and to creating a material that does not melt at the follow-on processing temperatures (e.g., cooking temperatures). The water content has found to be ideal for chewing quality and digestibility when set between 35% and 75%. This combination of the pressure, temperature and water content utilizing this technology is unique because typical dairy protein gels with water content at this level easily distort and melt.

Novel stable, edible compositions were developed utilizing liquid and powdered ruminant milk as a starting material. Each composition represents combinations of material characteristics important for the end use including texture and force strength, moisture level, and the ability to be shaped and/or molded. The compositions were quantified with physical peak compression strength measurements at associated temperature with specific total moisture, and a physically identifiable molded shape.

The novel composite structures were made to shape resemble animal, poultry, fish and crustacean flesh. They also have the identifiable shape, moisture and texture characteristics of the flesh that they resemble.

The disclosure features a natural ruminant milk derived solid composite with a peak compression strength range between 2 to 20 lbf when at a temperature of 50 to 80 degrees Fahrenheit with a total moisture between 30-75 percent. The starting material—liquid ruminant milk—is fluid with a measurable viscosity parameter between 1 and 250 centipoise. The composite uses include human and pet food. The composite can be frozen and heated and used in cooking processes including frying, sauté, grilling, sous vide, smoking, pressure cooking, baking, retorting, sublimation, vacuum drying, and/or conventional dehydration with the resulting product maintaining its physical molded characteristics without distortion such as softening or melting.

It is an objective of the present disclosure to develop non-animal, high-protein meat and seafood substitute composites that do not distort or melt under pressures up to 15 PSI and/or extreme temperatures ranging from 125 F to 700 F. These parameters have been tested to ensure the final cooking preparations will yield the appropriate material under pressure or extreme temperature.

The composites are novel, cost effective, sustainable, chemical free, edible proteins that can be used to economically and nutritionally replace beef, pork, poultry, fish & crustacean meats made from highly abundant liquid and powdered ruminant milk. The supply chain for the starting material is ubiquitous. The composites are an excellent protein source that can be complimented with added vitamins, minerals and bioactive substances. The composites have specific qualities including texture and shape that are at minimum equal to the cooked versions of the animal, poultry, porcine, fish, and crustacean it is replacing or being added to as an extender. Therefore the parameters of this disclosure led to the creation of products with minimum chemistry and natural food ingredients that yield precise material characterization with measured analysis.

An advantage of the disclosure starts with the use of ruminant milk as a feedstock. For an edible protein that is to be used as a meat and seafood alternative it wasn't a logical starting material, but it is highly pragmatic, sustainable, and compositionally superior to all other vegetarian low-carbon sources. As a base material it has been relied upon for feeding all forms of life and provides essential high quality amino acids versus low quality amino acids from plant proteins. It competes directly with expensive and unsustainable animal meat resources nutritionally. Then add to that fact the planet's fisheries are not sustainable for the long term. Therefore alternative edible protein structures that serve as meats and seafood feedstocks are a long term requirement.

There are hurdles. All meat, as we perceive it, has an identifiable texture, shape, and appearance. These factors are the targets for measurements of composites produced in accordance with the present disclosure.

All examples and features mentioned below can be combined in any technically possible way.

In one aspect, an edible, ruminant-milk-derived solid composition includes at least 30% by weight processed milk from at least one ruminant animal, and having a compression strength of from about 1.5 to about 11.5 lbf (e.g., from 1.47 lbf to 11.11 lbf) when measured at from 60 to about 85 degrees Fahrenheit (e.g., from 61.9 F to 83.5 F), and a moisture content of from about 40% to about 75% (e.g., from 38% to 75%).

Some examples include one of the above and/or below features, or any combination thereof. In an example the composition is used as a textured edible substitute for beef, pork, poultry, fish, and crustacean. In an example the composition is made utilizing thermal process molding temperatures of from 100 F-300 F. In an example the composition is heat treated at temperatures between 100 F-700 F. In an example the composition is made utilizing pressure between 0.1-50 PSI.

Some examples include one of the above and/or below features, or any combination thereof. In an example the composition is made utilizing liquid ruminant animal milk or milk mixtures with a viscosity between 1-250 cP. In an example the composition is made utilizing a milk mixture comprising rehydrated dried milk powder from ruminant animals. In an example the composition is made using a mixture of liquid milk and dried milk powder from ruminant animals. In an example the composition comprises more than 10% ruminant milk derived protein and fat based on a 0% moisture basis. In an example the composition is derived from at least one of liquid and dry ruminant milk. In an example the ruminant animals comprise at least one of dairy cows, water buffalo, goats, sheep, and camels. In an example the milk comprises casein, whey protein and calcium.

Some examples include one of the above and/or below features, or any combination thereof. In an example the composition further includes less than 70% by weight (dry basis) other materials selected from the group of materials consisting of grain products, flavors, spices, chlorides, vitamins, minerals, fatty acids, bacteria, enzymes, complimentary proteins including textured vegetable protein, fibers, and carbohydrates. In an example the other materials comprise at least one of Glucomannan, Carrageenan, Alginate, Gellan Gum, Xanthan Gum, Unmodified and/or Modified Starch, Transglutaminase, Rennet, Mesophilic Bacteria, Thermophilic Bacteria, Hydroxyproline, Flavors, Natural and Modified Yeast, Lactic Acid, Animal and Or Plant and or Bacterial Hemoglobin, Garlic, Pepper, Onion, Fungus and all Fungal Proteins, Herbs, Calcium and or Sodium and or Potassium Chloride, Cellulose, Lipase, Algae and Algae Oil, Fish Oil, Liquid and or Dry Egg and Egg White, Coconut Meat and or Fat, Corn Products, Rice Products, Wheat Flour and or Protein and or Starch Products, Millet, Flax, Soy Protein and Products, Pea Protein and Products, Sunflower Products, Rape Seed Products, Mung Bean Products, Fava Bean Products, Safflower Products, Olive Products, Gourds, Tomato Products, Beet Products, Tubers, Root Vegetables, Seed Meal and Proteins, Natural Coloring Agents, Red Rice Yeast, Casein Protein, Whey Protein, Lactose, Dairy Fat and or Butter, Cream, and Milk Solids, Palm Oil, Chymosin, and Vegetarian Rennet including Mucur Miehei.

Some examples include one of the above and/or below features, or any combination thereof. In an example the milk is processed using at least one of bacteria, enzymes, mesophilic bacteria, thermophilic bacteria, and rennet. In an example the composition further includes at least one of animal and poultry and fish and crustacean and shellfish and plant materials in a ratio with the processed milk between 10:1 and 1:10. In an example the plant materials comprise at least one of legumes, grains, tree nuts, fruits, and vegetables. In an example the animal and poultry are selected from the group consisting of ruminants, bovine, porcine, poultry, and fowl. In an example the composition is suitable for at least one of heated cooking, frying, sauté, grilling, sous vide, freezing, smoking, pressure cooking, sublimation, vacuum drying, boiling, steaming, baking, and dehydration.

In another aspect a method of producing an edible, ruminant-milk-derived solid composition, comprises heating milk from at least one ruminant animal, fermenting the milk after heating, further fermenting the fermented heated milk in a separate fermentation step, filtering after the separate fermentation step, and processing the filtrate at elevated temperature and pressure, to create a composite edible material that has a compression strength of from about 1.5 to about 11.5 lbf when measured at from 60 to about 85 degrees Fahrenheit, and a moisture content of from about 40% to about 75%.

### DETAILED DESCRIPTION

The present disclosure involves the conversion of liquid and dried ruminant milk into molded and shaped edible protein compositions resembling the flesh of bovine, porcine, poultry, fish and crustacean. The compositions can be processed like the meats/seafoods that they resemble, e.g., baking, grilling, frying, steaming and the like, as further disclosed herein. Accordingly, the subject composite materials look like, have the texture of, and can be cooked like the meat or seafood that they mimic.

Examples of the compositions and methods discussed herein are not limited in application to the details set forth in the following description as they are capable of implementation in other examples and of being practiced or of being carried out in various ways. Examples of specific implementations are provided herein for illustrative purposes only and are not intended to be limiting. In particular, functions, components, elements, and features discussed in connection with any one or more examples are not intended to be excluded from a similar role in any other examples.

Examples disclosed herein may be combined with other examples in any manner consistent with at least one of the principles disclosed herein, and references to “an example,” “some examples,” “an alternate example,” “various examples,” “one example” or the like are not necessarily mutually exclusive and are intended to indicate that a particular feature, structure, or characteristic described may be included in at least one example. The appearances of such terms herein are not necessarily all referring to the same example.

Also, the phraseology and terminology used herein is for the purpose of description and should not be regarded as limiting. Any references to examples, components, elements, acts, or functions of the compositions and methods herein referred to in the singular may also embrace embodiments including a plurality, and any references in plural to any example, component, element, act, or function herein may also embrace examples including only a singularity. Accordingly, references in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

The present disclosure targets the conversion of liquid and dried ruminant milk into molded and shaped edible protein compositions resembling the flesh of bovine, porcine, poultry, fish and crustacean. The following table (Table 1) illustrates relevant properties of the examples set forth below.

TABLE 1

| | Viscosity | | | |
|---|---|---|---|---|
| | of ruminant | | Compression | |
| | milk before | Temp/PSI | strength of | % H2O |
| | processing | of thermal | final composite | in final |
| Sample | (cP) | processing | (LBF/Temp (F.)) | composite |
| E1 | 100 | 225 F. @ 13.5 | 6.7 @ 73.5 | 67 |
| E2 | 100 | 225 F. @ 13.5 | 2.14 @ 73.0 | 71 |
| E3 | 5 | 150 @ 0.43 | 4.84 @ 76.0 | 70 |
| E4 | 12 | 150 @ 0.43 | 8.47 @ 83.5 | 65 |
| E5 | 10 | 150 @ 0.40 | 1.47 @ 81.5 | 75 |
| E6 | 10 | 225 F. @ 14.0 | 1.91 @ 81.5 | 72 |
| E7 | 10 | 225 F. @ 14.0 | 2.11 @ 79.5 | 72 |
| E8 | 105 | 225 @ 13.5 | 11.11 @ 73.0 | 70 |
| E9 | 125 | 225 @ 13.5 | 8.18 @ 61.9 | 57.5 |
| E10 | 3 | 150 @ 0.43 | 5.94 @ 76.7 | 70 |
| E11 | 125 | 225 @ 13.5 | 10.69 @ 76.2 | 38 |
| E12 | 105 | 225 @ 13.5 | 4.84 @ 69.2 | 51 |
| E13 | 105 | 225 @ 13.5 | 3.74 @ 73.2 | 52.6 |

### EXAMPLES

E1 is a Molded and Shaped Composite with Peak Compression Force of 6.7 LBF at 73.5 F with 67% total moisture. 3860× (i.e., parts by weight) Dairy Cow Whole Milk is blended with 270× Dry No Fat Milk Powder to form a liquid solution then heated to 220 F for 1 minute at 13 PSI then cooled to 110 F. 1.6× Mesophilic bacteria added in an ambient 50 ml reverse osmosis (R/O) solution. Product fermented in 110 F chamber for 1.5 hrs. 2.22× Rennet in ambient 50 ml R/O solution added and second fermentation conducted for 1.5 hrs. Fermentation product then filtered to remove the residual soluble co-product fats, carbohydrates and proteins, mixed with natural flavor and spices, and transferred to a rectangular mold and thermally processed for 8 minutes at 225 F at 13.5 PSI declining to 1.0 PSI for 10 minutes.

E2 is a Molded and Shaped Composite with Peak Compression Force of 2.14 LBF at 73 F with 71% total moisture. 3860× Dairy Cow Whole Milk is blended with 270× Dry No Fat Milk Powder to form a liquid solution then heated to 225 F for 1 minute at 13 PSI then cooled to 110 F. 1.6× Mesophilic bacteria added in an ambient 50 ml R/O solution. Product fermented in 110 F chamber for 1.5 hrs. 2.22× Rennet in ambient 50 ml R/O solution added and second fermentation conducted for 1.5 hrs. Fermentation product then filtered, mixed with natural flavor and transferred to a round mold and thermally processed for 120 minutes at 150 F at 0.4 PSI.

E3 is a Molded and Shaped Composite with Peak Compression Force of 4.85 LBF at 76 F with 70% total moisture. 5790× Goat Milk is heated to 180-195 F for 15 minutes then cooled to 110 F. 0.44× Mesophilic bacteria added in an ambient 50 ml R/O solution. 2.2× Calcium Chloride in ambient 50 ml R/O solution added. Product fermented in 110 F chamber for 1.0 hrs. 2.22× Rennet in ambient 50 ml R/O solution added and second fermentation conducted for 1.0 hrs. Fermentation product then filtered and transferred to an egg shaped silicone mold unit and thermally processed for 120 minutes at 150 F at 0.433 PSI.

E4 is a Molded and Shaped Composite with Peak Compression Force of 8.47 LBF at 83.5 F with 65% total moisture. 3860× Sheep Milk is heated to 180-195 F for 30 minutes then cooled to 110 F. 0.50× Mesophilic bacteria added in an ambient 50 ml R/O solution. Product fermented in 110 F chamber for 1.0 hrs. 2.25× Rennet in ambient 50 ml R/O solution added and second fermentation conducted for 1.0 hrs. Fermentation product then filtered and transferred to a hamburger shaped silicone mold and thermally processed for 120 minutes at 150 F at 0.433 PSI followed by 120 minutes at 200 F in propane drying-smoking unit.

E5 is a Molded and Shaped Composite with Peak Compression Force of 1.47 LBF at 81.5 F with 73% total moisture. 1930× Camel Milk is heated to 180-195 F for 15 minutes then cooled to 110 F. 0.50× Mesophilic bacteria added in an ambient 50 ml R/O solution. Product fermented in 110 F chamber for 1.0 hrs. 2.25× Rennet in ambient 50 ml R/O solution added and second fermentation conducted for 1.0 hrs. Fermentation product then filtered and transferred to a round shaped Teflon coated metal mold and thermally processed for 120 minutes at 150 F at 0.40 PSI followed by 8 minutes at 220 F at 13 PSI for 8 minutes.

E6 is a Molded and Shaped Composite with Peak Compression Force of 1.91 LBF at 81.5 F with 72% total moisture. 3,860× Water Buffalo milk is heated to 180-195 F for 15 minutes then cooled to 110 F. 0.50× Mesophilic bacteria added in an ambient 50 ml R/O solution. Product fermented in 110 F chamber for 1.0 hrs. 2.25× Rennet in ambient 50 ml R/O solution added and second fermentation conducted for 1.0 hrs. Fermentation product then filtered and mixed with natural flavors then transferred to a rectangular Teflon coated mold and thermally processed for 20 minutes at 225 F at 13 PSI followed by 60 minutes at 150 F at 0.40 PSI.

E7 is a Molded and Shaped Composite with Peak Compression Force of 2.11 LBF at 79.5 F with 72% total moisture. 3,860× Water Buffalo milk is heated to 180-195 F for 15 minutes then cooled to 110 F. 0.50× Mesophilic bacteria added in an ambient 50 ml R/O solution. Product fermented in 110 F chamber for 1.0 hrs. 2.25× Rennet in ambient 50 ml R/O solution added and second fermentation conducted for 1.0 hrs. Fermentation product then filtered and mixed with natural flavors then transferred to a rectangular Teflon coated mold and thermally processed for 20 minutes at 225 F at 13 PSI followed by 60 minutes at 150 F at 0.40 PSI.

E8 is a Molded and Shaped Composite with Peak Compression Force of 11.11 LBF at 73.0 F with 70% total moisture. 1930× Dairy Cow Whole Milk is blended with 270× Dry No Fat Milk Powder to form a liquid solution then heated to 220 F for 1 minute at 13 PSI then cooled to 110 F. 1.6× Mesophilic bacteria added in an ambient 50 ml R/O solution. Product fermented in 110 F chamber for 1.5 hrs. 2.22× Rennet in ambient 50 ml R/O solution added and second fermentation conducted for 1.5 hrs. Fermentation product then filtered, mixed with natural flavor and spices and transferred to a rectangular mold and thermally processed for 8 minutes at 225 F at 13.5 PSI declining to 1.0 PSI for 10 minutes.

E9 is a Molded and Shaped Composite with Peak Compression Force of 8.18 LBF at 61.9 F with 57.54% total moisture. 7720× Dairy Cow Whole Milk is blended with 1816× Dry No Fat Milk Powder to form a liquid solution then heated to 115 F for 1 minute then cooled to 110 F. 4.06× Mesophilic bacteria, 12.5× lipase, 10 g rennet, 10 g calcium chloride added in an ambient 200 ml R/O solution. Product fermented in 110 F chamber for 3.0 hrs. Fermentation product then filtered transferred to 5 round metallic Teflon coated molds and thermally processed for 80 minutes at 225 F at 13.5 PSI declining to 1.0 PSI for 30 minutes.

E10 is a Molded and Shaped Composite with Peak Compression Force of 5.94 LBF at 76.7 F with 70% total moisture. 5790× Goat Milk is heated to 185 F for 30 minutes then cooled to 110 F. 1.7× Mesophilic bacteria added in an ambient 50 ml R/O solution. 2.2× Calcium Chloride in ambient 50 ml R/O solution added. Product fermented in 110 F chamber for 1.0 hrs. 2.22× Rennet in ambient 50 ml R/O solution added and second fermentation conducted for 1.5 hrs. Fermentation product then filtered and transferred to a rectangular silicone vacuum mold and thermally processed for 120 minutes at 150 F at 0.433 PSI.

E11 is a Molded and Shaped Composite with Peak Compression Force of 10.69 LBF at 76.2 F with 38% total moisture. 7720× Dairy Cow Whole Milk is blended with 1816× Dry No Fat Milk Powder to form a liquid solution then heated to 115 F for 1 minute then cooled to 110 F. 4.06× Mesophilic bacteria, 12.5× lipase, 10 g rennet, 10 g calcium chloride added in an ambient 200 ml R/O solution. Product fermented in 110 F chamber for 3.0 hrs. Fermentation product then filtered transferred to 5 large round metallic Teflon coated molds and thermally processed for 80 minutes at 225 F at 13.5 PSI declining to 1.0 PSI for 30 minutes followed by 240 minutes drying at 110 F.

E12 is a Molded and Shaped Composite with Peak Compression Force of 4.84 LBF at 69.2 F with 51% total moisture. 7720× Dairy Cow Whole Milk is blended with 600× Dry No Fat Milk Powder to form a liquid solution then heated to 115 F for 1 minute then cooled to 110 F. 2.0× Mesophilic bacteria, 4.39× lipase, 4.0 g rennet, 5.2 g calcium chloride, 3.0× transglutamase added in an ambient 250 ml R/O solution. Product fermented in 110 F chamber for 3.0 hrs. Fermentation product then filtered transferred to 5 large round metallic Teflon coated molds and thermally processed for 80 minutes at 225 F at 13.5 PSI declining to 1.0 PSI for 30 minutes followed.

E13 is a Molded and Shaped Composite with Peak Compression Force of 3.74 LBF at 73.2 F with 52.6% total moisture. 7720× Dairy Cow Whole Milk is blended with 600× Dry No Fat Milk Powder to form a liquid solution then heated to 115 F for 1 minute then cooled to 110 F. 2.0× Mesophilic bacteria, 4.39× lipase, 4.0 g rennet, 5.2 g calcium chloride, 3.0× transglutamase added in an ambient 250 ml R/O solution. Product fermented in 110 F chamber for 3.0 hrs. Fermentation product then filtered transferred to 5 large round metallic Teflon coated molds and thermally processed for 10 minutes at 225 F at 13.5 PSI declining to 1.0 PSI for 30 minutes.

Having described above several aspects of at least one example, it is to be appreciated various alterations, modifications, and improvements will readily occur to those skilled in the art. Such alterations, modifications, and improvements are intended to be part of this disclosure and are intended to be within the scope of the invention. Accordingly, the foregoing description and drawings are by way of example only, and the scope of the invention should be determined from proper construction of the appended claims, and their equivalents.

What is claimed is:

1. An edible, ruminant-milk-derived solid composition, comprising at least 30% by weight processed milk from at least one ruminant animal, and having a compression strength of from about 1.5 to about 11.5 lbf when measured at from 60 to about 85 degrees Fahrenheit, and a moisture content of from about 40% to about 75%.

2. The composition of claim 1 used as a textured edible substitute for beef, pork, poultry, fish, and crustacean.

3. The composition of claim 1 made utilizing thermal process molding temperatures of from 100 F-300 F.

4. The composition of claim 3 further comprising heat treating at temperatures between 100 F-700 F.

5. The composition of claim 1 made utilizing pressure between 0.1-50 PSI.

6. The composition of claim 1 made utilizing liquid ruminant animal milk or milk mixtures with a viscosity between 1-250 cP.

7. The composition of claim 6 made utilizing a milk mixture comprising rehydrated dried milk powder from ruminant animals.

8. The compositions in claim 6 comprising a mixture of liquid milk and dried milk powder from ruminant animals.

9. The composition of claim 1 comprising more than 10% ruminant milk derived protein and fat based on a 0% moisture basis.

10. The composition of claim 6 derived from at least one of liquid and dry ruminant milk.

11. The composition of claim 1 wherein the ruminant animals comprise at least one of dairy cows, water buffalo, goats, sheep, and camels.

12. The composition of claim 1 wherein the milk comprises casein, whey protein and calcium.

13. The composition of claim 1 further comprising less than 70% by weight (dry basis) other materials selected from the group of materials consisting of grain products, flavors, spices, chlorides, vitamins, minerals, fatty acids, bacteria, enzymes, complimentary proteins including textured vegetable protein, fibers, and carbohydrates.

14. The composition of claim 13 wherein the other materials comprise at least one of Glucomannan, Carrageenan, Alginate, Gellan Gum, Xanthan Gum, Unmodified and/or Modified Starch, Transglutaminase, Rennet, Mesophilic Bacteria, Thermophilic Bacteria, Hydroxyproline, Flavors, Natural and Modified Yeast, Lactic Acid, Animal and Or Plant and or Bacterial Hemoglobin, Garlic, Pepper, Onion, Fungus and all Fungal Proteins, Herbs, Calcium and or Sodium and or Potassium Chloride, Cellulose, Lipase, Algae and Algae Oil, Fish Oil, Liquid and or Dry Egg and Egg White, Coconut Meat and or Fat, Corn Products, Rice Products, Wheat Flour and or Protein and or Starch Products, Millet, Flax, Soy Protein and Products, Pea Protein and Products, Sunflower Products, Rape Seed Products, Mung Bean Products, Fava Bean Products, Safflower Products, Olive Products, Gourds, Tomato Products, Beet Products, Tubers, Root Vegetables, Seed Meal and Proteins, Natural Coloring Agents, Red Rice Yeast, Casein Protein, Whey Protein, Lactose, Dairy Fat and or Butter, Cream, and Milk Solids, Palm Oil, Chymosin, Vegetarian Rennet including Mucur Miehei.

15. The composition of claim 1 wherein the milk is processed using at least one of bacteria, enzymes, mesophilic bacteria, thermophilic bacteria, and rennet.

16. The composition of claim 1 further comprising at least one of animal and poultry and fish and crustacean and shellfish and plant materials in a ratio with the processed milk between 10:1 and 1:10.

17. The composition of claim 16 wherein the plant materials comprise at least one of legumes, grains, tree nuts, fruits, and vegetables.

18. The composition of claim 16 wherein the animal and poultry are selected from the group consisting of ruminants, bovine, porcine, poultry, and fowl.

19. The composition of claim 1 that is suitable for at least one of heated cooking, frying, sauté, grilling, sous vide, freezing, smoking, pressure cooking, sublimation, vacuum drying, boiling, steaming, baking, and dehydration.

20. A method of producing an edible, ruminant-milk-derived solid composition, comprising heating milk from at least one ruminant animal, fermenting the milk after heating, further fermenting the fermented heated milk in a separate fermentation step, filtering after the separate fermentation step, and processing the filtrate at elevated temperature and pressure, to create a composite edible material that has a compression strength of from about 1.5 to about 11.5 lbf when measured at from 60 to about 85 degrees Fahrenheit, and a moisture content of from about 40% to about 75%.
